(19) United States
(12) Patent Application Publication
(10) Pub. No.: US 2007/0134181 A1
(43) Pub. Date: Jun. 14, 2007

(54) Cosmetic composition comprising an ester of dimerdilinoleic acid and of polyol(s) and a silicone surfactant

(75) Inventors: Momoko Shimizu, Tokyo (JP); Emiko Tokunaga, Kawasaki-city (JP)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/634,891

(22) Filed: Dec. 7, 2006

Related U.S. Application Data
(60) Provisional application No. 60/754,190, filed on Dec. 28, 2005.

(30) Foreign Application Priority Data
Dec. 8, 2005 (FR) 05 53798

(51) Int. Cl. A61K 8/37

(52) U.S. Cl. 424/64; 424/70.31

(57) ABSTRACT

The present invention relates to an anhydrous cosmetic composition for caring for and/or making up the skin and/or the lips, comprising at least one ester of dimerdilinoleic acid and of polyol(s) or an ester thereof, the viscosity of which, measured at about 25° C., is greater than or equal to about 20 000 mPa·s, at least one film-forming agent and at least one volatile oil.

This non provisional application claims the benefit of French Application No. 05 53798 filed on Dec. 8, 2005 and U.S. Provisional Application No. 60/754,190 filed on Dec. 28, 2005.

The present invention relates to cosmetic compositions for caring for and/or making up the skin and/or the lips, comprising an ester of dimerdilinoleic acid and of polyol(s) or an ester thereof, a film-forming agent and a volatile oil.

The compositions according to the invention may show improved gloss and/or colour staying power.

The present invention also relates to a process for making up the human face and/or body using a composition according to the invention.

A composition according to the invention may be a makeup and/or care product intended to be applied to the body, for example the face, and/or the lips, and may for example be a lipstick, a lip balm, a lip pencil, a liquid or solid foundation, for example cast as a stick or a dish, a concealer product, a skin colouring product, an eye makeup product, for instance an eyeliner, for example in pencil form, a mascara for example in cake form, or alternatively an eyeshadow or a lip gloss.

Many cosmetic compositions exist for which gloss properties and colour effect properties of the deposited film, after application to the skin and/or the lips, are desired. These properties generally contribute towards the desired aesthetic effect.

Moreover, it is also required that this aesthetic effect be maintained over time, for instance in the face of the various external factors liable to affect its gloss and/or colour properties.

In order to give cosmetic compositions good gloss properties and for instance good gloss staying power, it has been proposed to use “glossy” oils such as oily polymers, for instance high-viscosity polybutenes, esters of fatty acids or of fatty alcohols with a high carbon number (typically greater than 16), or alternatively certain plant oils.

However, these compounds may under certain circumstances be tacky on application and/or over time, which may result in considerable unpleasant sensations and a feeling of discomfort for the user.

The inventors have been able to observe, surprisingly, that the use of certain esters of dimerdilinoleic acid and of polyol(s), or an ester thereof, in combination with a film-forming agent, for instance an MQ resin or a PSPA polymer, to prepare cosmetic compositions, makes it possible to give these compositions improved colour staying power without affecting their combination of aesthetic properties, for example the gloss effect and the gloss staying power, while at the same time affording a comfortable sensation on application.

For example, a cosmetic composition according to the invention may have the advantageous property of not causing any tacky sensation after application.

Thus, according to one of these aspects, one subject of the present invention is an anhydrous cosmetic composition for caring for and/or making up the skin and/or the lips, comprising at least one ester of dimerdilinoleic acid and of polyol(s) or an ester thereof, the viscosity of which, measured at about 25° C., is greater than or equal to about 20 000 mPa·s, at least one film-forming agent and at least one volatile oil.

For the purposes of the invention, the term “anhydrous composition” means a composition containing less than 5% by weight, less than 3% by weight, less than 2% by weight or less than 1% by weight of water relative to the total weight of the composition.

According to another of its aspects, a subject of the present invention is a cosmetic composition for caring for and/or making up the skin and/or the lips, comprising at least one ester of general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15,
   at least one film-forming agent and at least one volatile oil.

According to another of its aspects, a subject of the present invention is a cosmetic composition for caring for and/or making up the skin and/or the lips comprising at least one ester of general formula (II) below: 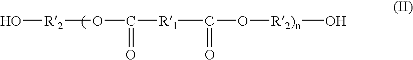
in which:
n is an integer ranging from 1 to 15,
OCOR′₁CO represents a dimerdilinoleate residue,
OR′₂O represents a diglyceryl residue of general formula (III) below: 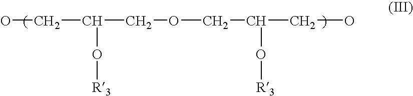
in which:
   R′₃ represents H or OR′₃ represents a fatty acid residue, at least one film-forming agent and at least one volatile oil.

According to another of its aspects, a subject of the present invention is the use of at least one ester of dimerdilinoleic acid and of polyol(s) or an ester thereof, according to the invention, in combination with at least one volatile oil and at least one film-forming agent, for the preparation of a cosmetic care and/or make-up composition with improved mean colour staying power.

According to another of its aspects, a subject of the present invention is for a method of preparation of a cosmetic composition for caring for and/or making up the skin and/or the lips, which has improved colour staying power.

According to another of its aspects, a subject of the present invention is for a method for the preparation of a cosmetic composition for caring for and/or making up the skin and/or the lips, which has improved colour staying power, comprising at least a step of combining at least one ester of dimerdilinoleic acid and of polyol(s), or an ester thereof, the viscosity of which, measured at 25° C., is greater than or equal to 20 000 mPa·s, with at least one volatile oil and at least one film-forming agent. According to yet another of its aspects, a subject of the present invention is a process for making up and/or caring for the skin and/or the lips, comprising at least one step that consists in applying a composition in accordance with the invention to at least part of a support.

According to one of its advantages, the present invention makes it possible to provide cosmetic compositions whose gloss staying power and/or gloss is not affected, or is even improved, while at the same time showing improved colour staying power.

According to another of its advantages, the present invention makes it possible to provide compositions whose colour staying power is improved over time, for instance with regard to various external attacking factors, such as meals or contact with fabrics.

According to another of its advantages, the present invention makes it possible to provide cosmetic compositions that do not cause any sensation of tack or discomfort when applied.

Ester of Dimerdilinoleic Acid and of Polyol(s)

In the expression “ester of dimerdilinoleic acid and of polyol(s) or an ester thereof”, the term “or an ester thereof” is intended to denote one of the derivatives of these dimerdilinoleic acid esters of polyol(s) obtained either by reaction of alcohol function(s) of the polyol, which are not employed in bonds of ester type with acid functions of the dilinoleic acid, with one or more carboxylic functions of acid molecules other than dilinoleic acid or alternatively by reaction of acid function(s) of the dilinoleic dimer, which are not employed in bonds of ester type with alcohol functions of the polyol, with alcohol functions of alcohol molecules other than the polyol.

Dimerdilinoleic Acid

The dimerdilinoleic acid that is suitable for use in the present invention may be obtained by polymerization reaction, for example by intermolecular dimerization of at least one linoleic acid.

The oxidation stability of the compound may be improved by hydrogenating the double bonds remaining after the dimerization reaction.

The linoleic acid dimer may also be obtained by dimerization of the hydrogenated form of linoleic acid.

The hydrogenated form of the acid or of the diacid may be partial or total, and may correspond, for example, to the saturated form, which is more oxidation-stable.

As indicated previously, the carboxylic functions of the dimerdilinoleic acid residue not engaged in the ester bond with the polyol residue(s) may be engaged in other ester bonds with other alcohol functions of alcohol molecules other than the polyol(s).

These alcohol molecules or residues may be monoalcohols or polyols.

As examples of alcohol residues that are suitable for use in the invention, mention may be made of hydrocarbon-based compounds comprising a hydroxyl function and containing from 4 to 40 carbon atoms, for example from 6 to 36 carbon atoms, for example from 8 to 32 carbon atoms, for example from 16 to 28 carbon atoms and for example from 18 to 24 carbon atoms.

As examples of monoalcohols that are suitable for the invention, mention may be made, in a non-limiting manner, of butanol, pentanol, propanol, hexanol, heptanol, octanol, decanol, dodecanol, hexadecanol, octadecanol, eicosadecanol, phytosterol, isostearol, stearol, cetol, behenol, etc.

Polyols

The term “polyol” is intended to denote any hydrocarbon-based compound comprising at least two hydroxyl functions and containing from 4 to 40 carbon atoms, for example from 6 to 36 carbon atoms, for example from 8 to 32 carbon atoms, for example from 16 to 28 carbon atoms and for example from 18 to 24 carbon atoms.

The hydrocarbon-based chains may be interrupted, where appropriate, by the presence of at least one hetero atom, and for example an oxygen atom.

A polyol or a polyol ester that is suitable for use in the present invention may comprise, for example, from 2 to 12 hydroxyl functions, for example from 2 to 8 hydroxyl functions, and for example from 4 to 6 hydroxyl functions.

Where appropriate, the hydroxyl functions, other than those already employed in an ester bond with the dimerdilinoleic acid, may also be employed, wholly or partly with other ester bonds via reactivity with acid molecules other than the dimerdilinoleic acid.

The polyol or an ester thereof that is suitable for use in the present invention may be chosen for example from linear, branched, cyclic or polycyclic, saturated or unsaturated alcohols.

Thus, the polyol may be chosen, for example, from a diol, a triol, a tetraol, or a pentaol, or an ester thereof.

The polyol may be a diol, or an ester thereof, chosen for example from a fatty alcohol dimer, a monoglycerol or polyglycerol, a C₂₋₄ monoalkylene or polyalkylene glycol, 1,4-butanediol and pentaerythritol.

As examples of diols that are also suitable for use in the invention, mention may be made, in a non-exhaustive manner, of butanediol, pentanediol, propanediol, hexanediol, hexylene glycol, heptanediol, octanediol, nonanediol, decanediol, 1-decanediol, dodecanediol, tridecanediol, tetradecanediol, pentadecanediol, hexadecanediol, nonadecanediol, octadecanediol, cyclohexanediol, diglycerol, erythritol, pentaerythritol, xylitol, sorbitol, ethylene glycol and xylene glycol, and isomers thereof.

A fatty alcohol dimer may also be the product of hydrogenation, for example catalytic hydrogenation, of a fatty acid dimer, which is itself obtained by dimerization of at least one unsaturated fatty acid, for example of C₈ to C₃₄, for example of C₁₂ to C₂₂, for example of C₁₆ to C₂₀ or for example of C₁₈.

A fatty alcohol dimer may range from C₁₆ to C₆₈, for example from C₂₄ to C₄₄, from C₃₂ to C₄₀, or may be of C₃₆.

According to one embodiment, the fatty alcohol dimer may be a diol dimer deriving from the hydrogenation of dilinoleic diacid. It is generally in a saturated form.

A fatty alcohol dimer may be, for example, a dilinoleol dimer.

As an example of a diol that may be suitable for use in the invention, mention may be made for example of diglycerol.

This compound is a glycerol dimer resulting from the condensation of two molecules of glycerol, with the loss of a water molecule.

The term “diglycerol” denotes any isomer combination that can result from such a condensation, for instance linear isomers, branched isomers and, where appropriate, cyclic isomers resulting from an intramolecular dehydration of a diglycerol molecule.

The diglycerol may be obtained via any process known to those skilled in the art and for example those described in patent EP 0 750 848.

As examples of acid molecules that can interact with one or more hydroxyl functions of the polyol, not employed in the ester bond with the dimerdilinoleic acid, mention may be made, in a non-limiting manner, of molecules derived from isostearic acid, behenic acid, phytosteric acid, stearic acid or cetylic acid.

An ester that is suitable for use in the present invention may be obtained by reacting a polyol or an ester thereof with a dimerdilinoleic acid, in a molar ratio of about 1.0:0.2-1.0.

An ester that may be suitable for use in the present invention may for example be obtained by reacting a dimerdilinoleic acid with a dilinoleol and, where appropriate, at least one additional monoalcohol chosen for example from behenol, isostearol, phytosterol, stearol and cetol, and mixtures thereof.

Thus, an ester used in the context of the present invention may be used in the form of a mixture of various esters, for example.

An ester that is suitable for the invention may be obtained, for example, by reacting a glycerol, an isostearic acid and a dimerdilinoleic acid, for example, in a molar ratio of 1.0:0.2-1.0:0.5-0.9.

As examples of esters of dimerdilinoleic acid and of polyol(s) or an ester thereof suitable for the invention, mention may be made of the esters described in patent applications JP 2004-256515 and JP 2005-179377.

An ester of dilinoleic acid and of polyol(s) or an ester thereof suitable for use in the present invention may have a molecular weight ranging from about 2000 to about 25 000 g/mol, for example from about 4000 to about 20 000 g/mol, for example from about 7000 to about 15 000 g/mol or for example from about 8000 to about 10 000 g/mol.

According to one embodiment, an ester in accordance with the invention may comprise an alternating sequence of dimerdilinoleate residue(s) and of residue(s) related to the said polyol(s), and for instance to the said diol(s), the said polyol(s) or diol(s) being, for example, as defined above.

Thus, in such a configuration, each of the two ends of the said sequence may bear, respectively, a unit OR′ and OR″ with R′ and R″ representing, independently of each other, a hydrogen atom or OR′ and OR″ representing, independently of each other, a C₂ to C₃₆, for example C₈ to C₂₄, for example C₁₂ to C₂₀ or for example C₁₆ to C₁₈ hydrocarbon-based monoalcohol residue.

According to one embodiment, R′ and R″ may both represent a hydrogen atom.

According to one embodiment, OR′ and OR″ may both represent an identical or different hydrocarbon-based monoalcohol residue.

As examples of hydrocarbon-based monoalcohol residues OR′ and OR″ that may be suitable for the invention, mention may be made of fatty alcohol residues.

According to one embodiment, an ester of dimerdilinoleic acid and of polyol(s) or an ester thereof that may be suitable for use in the present invention may have the general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue, that may range from C₁₆ to C₆₈, from C₂₄ to C₄₄, from C₃₂ to C₄₀, or may be of C₃₆,
OR₃ represents a monoalcohol residue that may range from C₄ to C₄₀, for example from C₆ to C₃₆, for example from C₈ to C₃₂, for example from C₁₆ to C₂₈, or for example from C₁₈ to C₂₄, and
n is an integer ranging from 1 to 15, for example from 2 to 10 or for example from 5 to 7.

According to one embodiment, OR₂O may represent a dimerdilinoleyl residue.

Moreover, OR₃ may represent a hydrocarbon-based monoalcohol residue chosen, for example, from behenyl, isostearyl and phytosteryl residues, and mixtures thereof.

According to another embodiment, the ester of dimerdilinoleic acid and of polyol(s) or an ester thereof that may be suitable for use in the invention may, for example, have the general formula (II) below: 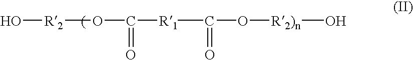
in which:
n is an integer ranging from 1 to 15, for example from 2 to 10 or for example from 5 to 7,
OCR′₁CO represents a dimerdilinoleate residue,
OR′₁O represents a diglyceryl residue of general formula (III) below: 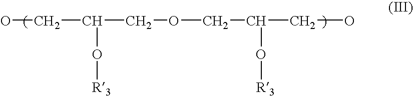
in which:
   R′₃ represents H or OR′₃ represents a fatty acid residue, that may range from C₈ to C₃₄, for example from C₁₂ to C₂₂, for example from C₁₆ to C₂₀, or for example may be of C₁₈.

According to one embodiment, the fatty acid residue represented by OR′₃ may be an isostearyl residue.

The viscosity of an ester of dimerdilinoleic acid and of polyol(s) or an ester thereof, according to the invention, may be measured according to any process known to those skilled in the art, and for example according to the conventional process described hereinbelow.

The viscosity may be measured using a cone/plate or parallel plate viscometer of Ares type (TA-Instrument) operating in kinetic sweep mode over a shear range of about 1-1000 s⁻¹ to induce a flow tension at about 1000 Pa.

The cone/plate or parallel plates may consist of a material selected from the group constituted of stainless steel, acrylic resins or polyphenylene sulfide (PPS resin).

The cone/plate diameter may be 25 mm (cone angle 0.10 radiants).

The measurement is performed at about 25° C.

Before any measurement, the stability of the sample is checked by means of the dynamic sweep period test, which makes it possible to determine if the sample is stable per se.

The shear viscosity is determined using the ETA value in the plateau region according to the flow.

The dynamic sweep period is determined at a frequency of 1.0 Hz over a period of 600 seconds.

The measurements at constant sweep rate are performed with a rate ranging from 1.0 to 1000 s⁻¹ and for example from 1.0 to 100 s⁻¹.

The viscosity of an ester of dimerdilinoleic acid and of polyol or an ester thereof suitable for use in the invention may range from about 20 000 mPa·s to about 150 000 mPa·s, for example from about 40 000 mPa·s to about 100 000 mPa·s and for example from about 60 000 mPa·s to about 80 000 mPa·s.

An ester that is suitable for the invention may be chosen for example from the esters having the following INCI nomenclature: polyglyceryl-2 isostearate dimerdilinoleate copolymer, bis-behenyl/isostearyl/phytosteryl dimerdilinoleyl dimerdilinoleate, and mixtures thereof.

Such compounds may be obtained, for example, under the reference Hailucent ISDA (Kokyu Alcohol) and Plandool-G (Nippon Fine Chemical Company Ltd).

An ester of dimerdilinoleic acid and of polyol(s) or an ester thereof suitable for use in the invention may be advantageously present in the cosmetic compositions according to the invention in an amount sufficient to give these compositions improved cosmetic properties, for instance in terms of gloss staying power.

The ester may be present in a content ranging from about 0.1% to about 80%, for example from about 2% to about 70% and for example from about 30% to about 50% by weight relative to the total weight of the composition.

Film-Forming Agent

A cosmetic composition according to the invention contains at least one film-forming agent that may be chosen for example from film-forming polymers. Certain film-forming polymers may be gelling polymers.

A film-forming agent may or may not be combined, in a composition according to the invention, with at least one auxiliary film-forming agent.

For the purposes of the invention, the term “polymer” means a compound containing at least 2 repeating units and for example at least 3 repeating units.

The term “film-forming polymer” is intended to denote a polymer capable of forming, by itself or in the presence of an auxiliary film-forming agent, a macroscopically continuous film on a support, for instance on keratin materials, and for example a cohesive film and for example a film whose cohesive and mechanical properties are such that the said film may be isolated from the said support.

A film-forming polymer may be present in a cosmetic composition according to the invention in a content ranging from about 0.1% to about 60% by weight, for example ranging from about 0.5% to about 60% by weight, for example ranging from about 1% to about 50% by weight, for example ranging from about 1% to about 40% by weight, for example ranging from about 2% to about 30% by weight and for example ranging from about 5% to about 25% by weight of film-forming polymer relative to the total weight of the composition.

When introduced in sufficient amount, in combination with an ester of dimerdilinoleic acid and of polyol(s), or an ester thereof as defined above, and a volatile oil as defined below, the film-forming agent has for instance the advantage of being able to give an improvement in the gloss staying power or even also in the colour staying power to the cosmetic compositions according to the invention.

In one embodiment, a polymer that is suitable for the invention may be selected from the group constituted of film-forming polymers that are soluble in an organic liquid medium, film-forming polymers that are dispersible in an organic liquid medium, film-forming polymers in the form of aqueous dispersions of polymer particles, and water-soluble film-forming polymers, and mixtures thereof.

Thus, a polymer that is suitable for the invention may be selected from the group constituted of:
film-forming polymers that are soluble in an organic liquid medium, for example liposoluble polymers, when the organic liquid medium comprises at least one oil;
film-forming polymers that are dispersible in an organic liquid medium, for example polymers in the form of non-aqueous dispersions of polymer particles, preferably dispersions in silicone oils or hydrocarbon-based oils; in one embodiment, the non-aqueous polymer dispersions may comprise polymer particles stabilized at their surface with at least one stabilizer;
film-forming polymers in the form of aqueous dispersions of polymer particles, often known as “latices”; in this case, the composition may comprise an aqueous phase;
water-soluble film-forming polymers; in this case, the composition may comprise an aqueous phase,
and mixtures thereof.

As film-forming polymers that may also be used in a cosmetic composition according to the present invention, mention may be made of synthetic polymers, of free-radical type or of polycondensate type, polymers of natural origin such as those described in patent application FR 04/50540 filed on 18 Mar. 2004, and mixtures thereof.

Film-forming polymers that may also be mentioned more particularly include acrylic polymers and copolymers, polyurethanes, polyesters, polyamides, polyureas, cellulose-based polymers, for instance nitrocellulose, silicone polymers, for instance silicone resins and silicone-grafted acrylic polymers, and polyamide polymers and copolymers, and mixtures thereof.

Thus, a film-forming polymer that is suitable for the invention may be selected from the group constituted of the film-forming polymers described in patent application WO 04/028487.

Similarly, a film-forming polymer that is suitable for the invention may be a crosslinked silicone compound as described in patent applications US 2003/0103918 and US 2003/0049216.

A film-forming polymer that is suitable for the invention may also be selected from the group constituted of those described in the international patent application filed under No. PCT/FR03/02849.

A film-forming polymer that is suitable for the invention may also be a film-forming linear ethylenic block polymer. In general, a block polymer may comprise at least one first block and at least one second block with different glass transition temperatures (Tg), the said first and second blocks being linked together via an intermediate block comprising at least one constituent monomer of the first block and at least one constituent monomer of the second block.

A film-forming polymer that may be used in the invention may also be in the form of a dispersion of particles, which are preferably solid, of a grafted ethylenic polymer in a liquid fatty phase. Such a dispersion is for instance described in the international patent application filed under the filing No. PCT/FR03/03709.

Thus, such a grafted ethylenic polymer may comprise an ethylenic skeleton that is insoluble in the liquid fatty phase, and side chains that are covalently bonded to the said skeleton and are soluble in the said dispersion medium. Advantageously, the film-forming polymer may be a copolymer of acrylic nature and may more particularly comprise one or more monomers selected from the group constituted of 2-ethylhexyl acrylate, isobutyl acrylate and methyl acrylate, and/or copolymers thereof.

The copolymers may advantageously be soluble or dispersible in cosmetic media and for example in an aqueous, aqueous-organic or organic medium. It may thus be in the form of an aqueous particle dispersion or in the form of an oily particle dispersion.

According to one embodiment, a film-forming polymer that is suitable for the invention may be featured by at least one copolymer of acrylic block copolymer type containing at least one block of limited conformation and one block of variable conformation. These two blocks are mutually incompatible and are characterized by a different glass transition temperature (Tg).

These first and second blocks may be linked together via an intermediate segment comprising at least one constituent monomer of the first block and at least one constituent monomer of the second block.

Such copolymers are also denoted by the term “block polymer”.

Each block of the block polymer is derived from one type of monomer or from several different types of monomers.

This means that each block may consist of a homopolymer or a copolymer, this copolymer constituting the block that may in turn be random or alternating.

The possible intermediate segment comprising at least one constituent monomer of the first block and at least one constituent monomer of the second block of the polymer is generally itself a random polymer.

The block polymer may be obtained via solution free-radical polymerization according to the process described in patent application EP 1 411 069.

As indicated above, the block of variable conformation and the block of limited conformation have different glass transition temperatures.

The difference between the glass transition temperatures of the blocks of limited and variable conformation is generally greater than 20° C., preferably greater than 30° C. and better still greater than 40° C.

As stated previously, the block of variable conformation has a glass transition temperature of less than 30° C., for example between −100° C. and +25° C., for example between −90° C. and +20° C. and better still between −80° C. and 0° C.

The monomers that may be used preferred for the block of variable conformation are alkyl acrylates whose alkyl chain comprises from 1 to 10 or even from 1 to 4 carbon atoms, such as methyl acrylate, isobutyl acrylate and 2-ethylhexyl acrylate, and copolymers thereof.

In an embodiment, the proportion of the block of variable conformation whose Tg may be generally less than 30° C., or even less than 20° C., ranges from 5% to 75%, preferably from 15% to 50% and better still from 25% to 45% by weight, relative to the weight of the block polymer.

The block of limited conformation advantageously has a Tg of greater than or equal to 40° C., for example a Tg ranging from 40 to 120° C., for example greater than 50° C. and for example greater than 60° C.

The monomers that may be used for the block of limited conformation are methyl methacrylate, isobutyl methacrylate, isobornyl(meth)acrylate, trifluoroethyl methacrylate, styrene and (meth)acrylic acid, and copolymers thereof.

In an embodiment, the proportion of the block of limited conformation with a Tg greater than or equal to 40° C. ranges from 20% to 90%, for example from 30% to 80% and even better still from 50% to 70% by weight relative to the weight of the block polymer.

As non-limiting illustrations of block copolymers that are suitable for the invention, mention may be made the following variants of copolymers:
a block of limited conformation with a Tg of greater than or equal to 40° C., for example with a Tg ranging from 70 to 110° C., which is a methyl methacrylate/acrylic acid copolymer,
a block of variable conformation with a Tg of less than or equal to 20° C., for example ranging from 0 to 20° C., which is a methyl acrylate homopolymer.

According to an embodiment, the copolymer according to the invention may comprise:
a block of limited conformation with a Tg of greater than or equal to 40° C., for example ranging from 70 to 100° C., which is a methyl methacrylate/acrylic acid/trifluoroethyl methacrylate copolymer, and
a block of variable conformation with a Tg of less than or equal to 20° C., for example ranging from 0 to 20° C., which is a methyl acrylate homopolymer.

According to an embodiment, the copolymer according to the invention may comprise:
a block of limited conformation with a Tg of greater than or equal to 40° C., for example ranging from 85 to 115° C., which is an isobornyl acrylate/isobutyl methacrylate copolymer, and
a block of variable conformation with a Tg of less than or equal to 20° C., for example ranging from −85 to −55° C., which is a 2-ethylhexyl acrylate homopolymer.

According to an embodiment, the copolymer according to the invention may comprise:
a block of limited conformation with a Tg of greater than or equal to 40° C., for example ranging from 95 to 125° C., which is an isobornyl acrylate/isobornyl methacrylate copolymer, and
a block of variable conformation with a Tg of less than or equal to 20° C., for example ranging from −35 to −5° C., which is an isobutyl acrylate homopolymer.

According to an embodiment of the invention, the film-forming polymer may be a polymer of grafted ethylenic polymer type.

The term “ethylenic polymer” means a polymer obtained via polymerization of ethylenically unsaturated monomers.

The term “grafted polymer” means a polymer having a skeleton comprising at least one pendent side chain or a side chain located at the end of a chain, and preferably a pendent side chain.

The grafted polymer may have a weight-average molecular mass (Mw) of between 5000 and 300 000, for example between 10 000 and 300 000, for example between 20 000 and 200 000 and for example between 25 000 and 150 000 g/mol.

Advantageously, the grafted ethylenic polymer may comprise an ethylenic skeleton that is insoluble in the said liquid fatty phase, and side chains that are covalently bonded to the said skeleton and are soluble in the liquid fatty phase.

The grafted ethylenic polymer may be a non-crosslinked polymer. The polymer may be obtained via polymerization of monomers comprising only one polymerizable group.

The grafted ethylenic polymer may be obtained according to the process described in document WO 04/055081 via free-radical polymerization in an organic polymerization medium:
of at least one ethylenic monomer, for example of at least one acrylic monomer and for example of at least one additional non-acrylic vinyl monomer, to form the said insoluble skeleton; and
of at least one macromonomer comprising a polymerizable end group to form the side chains, the said macromonomer having a weight-average molecular mass of greater than or equal to 200 g/mol and the content of polymerized macromonomer representing from 0.05% to 20% by weight of the polymer.

According to one embodiment of the invention, the grafted ethylenic polymer may be a grafted acrylic polymer, i.e. a polymer derived from the polymerization of at least one acrylic monomer.

Among the acrylic monomers capable of forming the skeleton of the ethylenic polymer, mention may be made of methyl, ethyl, propyl, butyl or isobutyl(meth)acrylate; methoxyethyl or ethoxyethyl(meth)acrylate; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate; dimethylaminopropylmethacrylamide; (meth)acrylic acid; and salts thereof; and copolymers thereof.

The acrylic monomers may be selected from the group constituted of methyl acrylate, methoxyethyl acrylate, methyl methacrylate, 2-hydroxyethyl methacrylate, (meth)acrylic acid and dimethylaminoethyl methacrylate, and mixtures thereof.

As regards the macromonomers, they may comprise at one of the ends of the chain a polymerizable end group capable of reacting during the polymerization with the acrylic monomers and optionally additional vinyl monomers, to form the side chains of the grafted ethylenic polymer. The said polymerizable end group may for example be a vinyl or (meth)acrylate or (meth)acryloxy) group, and preferably a (meth)acrylate group.

The macromonomers may be selected from the group constituted of macromonomers whose homopolymer has a glass transition temperature (Tg) of less than or equal to 25° C., for example ranging from −100° C. to 25° C. and for example ranging from −80° C. to 0° C.

The macromonomers may generally have a weight-average molecular mass of greater than or equal to 200 g/mol, preferably greater than or equal to 300 g/mol, for example greater than or equal to 500 g/mol and for example greater than 600 g/mol.

The macromonomers may have a weight-average molecular mass (Mw) ranging from 200 to 100 000 g/mol, for example ranging from 500 to 50 000 g/mol, for example ranging from 800 to 20 000 g/mol and for example ranging from 800 to 10 000 g/mol.

These macromonomers may be linear or branched C₈-C₂₂ alkyl(meth)acrylate homopolymers and copolymers, or polyolefins containing an ethylenically unsaturated end group, for example containing a (meth)acrylate end group.

The first types of carbon-based macromonomer are described for example in patents EP 895 467 and EP 96 459 and in the article Gillman K. F., Polymer Letters, Vol. 5, pages 477-481 (1967).

Mention may be made of macromonomers based on poly(2-ethylhexyl acrylate) or on poly(dodecyl acrylate) containing mono(meth)acrylate end groups.

The second types of macromonomer are described for example in U.S. Pat. No. 5,625,005, which mentions ethylene/butylene and ethylene/propylene macromonomers containing a (meth)acrylate reactive end group.

Mention may be made of poly(ethylene/butylene) methacrylate, such as the product sold under the name Kraton Liquid L-1253 by Kraton Polymers.

Silicone macromonomers that may be used include monomethacryloxypropyl polydimethylsiloxanes such as those sold under the name PS560-K6 by the company United Chemical Technologies Inc. (UCT) or under the name MCR-M17 by the company Gelest Inc.

As grafted ethylenic copolymer that is particularly preferred, dispersed in a non-silicone liquid fatty phase, it is possible to use those obtained via polymerization:
of methyl acrylate and of polyethylene/polybutylene macromonomer containing methacrylate end groups (for example Kraton L-1253), for example in a solvent selected from the group constituted of isododecane, isononyl isononanoate, octyldodecanol, diisostearyl malate and a C₁₂-C₁₅ alkyl benzoate (such as Finsolv TN);
methoxyethyl acrylate and polyethylene/polybutylene macromonomer containing methacrylate end groups (for example Kraton L-1253), for example in isododecane;
methyl acrylate/methyl methacrylate monomers and poly-ethylene/polybutylene macromonomer containing methacrylate end groups (for example Kraton L-1253), for example in isododecane;
methyl acrylate/acrylic acid monomers and polyethylene/polybutylene macromonomer containing methacrylate end groups (for example Kraton L-1253), for example in isododecane;
methyl acrylate/dimethylaminoethyl methacrylate monomers and polyethylene/polybutylene macromonomer containing methacrylate end groups (for example Kraton L-1253), for example in isododecane;
methyl acrylate/2-hydroxyethyl methacrylate monomers and polyethylene/polybutylene macromonomer containing methacrylate end groups (for example Kraton L-1253), for example in isododecane.

As grafted acrylic polymer that may be used, dispersed in a silicone liquid fatty phase, it is possible to use those obtained via polymerization:
of methyl acrylate and of monomethacryloxypropylpolydimethylsiloxane macromonomer with a weight-average molecular weight ranging from 800 to 6000 g/mol, for example in decamethylcyclopentasiloxane or phenyl trimethicone;
of methyl acrylate, acrylic acid and monomethacryloxypropylpolydimethylsiloxane macromonomer with a weight-average molecular weight ranging from 800 to 6000 g/mol, for example in decamethylcyclopentasiloxane or phenyl trimethicone.

According to another form of the invention, the film-forming polymer may be selected from the group constituted of silicone polymers grafted with non-silicone organic monomers. These polymers may be liposoluble, lipodispersible, water-soluble or dispersible in aqueous medium, where appropriate.

Examples of polymers with a polysiloxane skeleton grafted with non-silicone organic monomers that are suitable for use in the present invention, and also the specific method for preparing them, are described for example in patent applications EP-A-0 582 152, WO 93/23009 and WO 95/03776.

According to one embodiment, a silicone film-forming polymer that may be suitable for use in the present invention may be a copolymer comprising carboxylate groups and polydimethylsiloxane groups.

As copolymers that may be used, examples that may be mentioned include copolymers of acrylic acid and of stearyl acrylate containing polydimethylsiloxane grafts, copolymers of stearyl methacrylate containing polydimethylsiloxane grafts, copolymers of acrylic acid and of stearyl methacrylate containing polydimethylsiloxane grafts, copolymers of methyl methacrylate, butyl methacrylate, 2-ethylhexyl acrylate and stearyl methacrylate containing polydimethylsiloxane grafts.

According to one embodiment, a composition according to the invention may also comprise at least one auxiliary film-forming agent.

Physiologically Acceptable Medium

A composition according to the invention comprises a physiologically acceptable medium.

The term “physiologically acceptable medium” is intended to denote a medium that is particularly suitable for the application of a composition according to the invention to the skin and/or the lips. The physiologically acceptable medium is generally suited to the nature of the support onto which the composition is to be applied, and to the aspect in which the composition is intended to be conditioned.

The physiologically acceptable medium may comprise an aqueous and/or water-soluble phase and/or a fatty phase.

According to one embodiment, the aqueous phase or the fatty phase may form the continuous phase of the composition.

This aqueous phase may, where appropriate, be thickened, gelled or structured by also incorporating therein a conventional aqueous gelling agent for example of mineral origin, for instance clay, and/or of organic origin, for instance an aqueous gelling polymer.

According to one embodiment, a composition in accordance with the invention may be in the form of a suspension, a dispersion, a solution, a gel or an emulsion, for instance an oil-in-water (O/W), water-in-oil (W/O) or multiple (W/O/W, polyol/O/W or O/W/O) emulsion, or in the form of a cream, a stick, a paste, a mousse, a dispersion of vesicles, for instance of ionic or nonionic lipids, a two-phase or multi-phase lotion or a powder.

For the purposes of the present invention, the emulsions contain a lipophilic phase and a hydrophilic phase, the latter phase not systematically being water.

Thus, the cosmetic compositions in accordance with the invention may be in the form of an anhydrous emulsion.

According to an embodiment, the composition may contain, for example, a continuous fatty phase, which may contain less than 10% by weight of water, for example less than 5% by weight of water or even less than 1% by weight of water relative to the total weight of the composition.

According to an embodiment, a cosmetic composition according to the invention may be anhydrous, i.e. may contain less than 5%, for example less than 3%, for example less than 2% and for example less than 1% by weight of water relative to the total weight of the composition. These compositions may be in the form of oily gels, oily liquids, pastes or sticks or alternatively in the form of a vesicular dispersion containing ionic and/or nonionic liquids.

Fatty Phase

A cosmetic composition in accordance with the present invention comprises a fatty phase comprising at least one volatile oil as described below.

It may also comprise a fatty phase selected from the group constituted of a liquid fatty phase, for instance non-volatile oils, and a fatty substance that is solid at room temperature (20-25° C.) and atmospheric pressure, and mixtures thereof.

The term “oil” means any fatty substance that is in liquid form at room temperature (20-25° C.) and at atmospheric pressure. The liquid fatty phase may also contain, in addition to oils, other compounds dissolved in the oils, such as gelling agents and/or structuring agents.

The oil(s) may be present in a proportion of from 0.1% to 99% by weight, from at least 1% to 90% by weight, for example from 5% to 70% by weight, for example from 10% to 60% by weight or even from 20% to 50% by weight relative to the total weight of the cosmetic composition according to the invention.

The volatile or non-volatile oils may be hydrocarbon-based oils for example of animal or plant origin, synthetic oils, silicone oils or fluoro oils, or mixtures thereof.

For the purposes of the present invention, the term “silicone oil” means an oil comprising at least one silicon atom, and for instance at least one Si—O group.

The term “hydrocarbon-based oil” means an oil mainly containing hydrogen and carbon atoms, and possibly oxygen, nitrogen, sulfur and/or phosphorus atoms.

Volatile Oils

A composition in accordance with the invention comprises at least one volatile oil.

For the purposes of the present invention, the term “volatile oil” means an oil (or non-aqueous medium) capable of evaporating on contact with the skin in less than one hour, at room temperature and at atmospheric pressure. The volatile oil is a volatile cosmetic oil, which is liquid at room temperature, for instance having a non-zero vapour pressure, at room temperature and atmospheric pressure, for example having a vapour pressure ranging from 0.13 Pa to 40 000 Pa (10⁻³ to 300 mmHg), for example ranging from 1.3 Pa to 13 000 Pa (0.01 to 100 mmHg) and for example ranging from 1.3 Pa to 1300 Pa (0.01 to 10 mmHg).

The volatile hydrocarbon-based oils may be selected from the group constituted of hydrocarbon-based oils containing from 8 to 16 carbon atoms, and for example branched C₈-C₁₆ alkanes (also known as isoparaffins), for instance isododecane (also known as 2,2,4,4,6-pentamethylheptane), isodecane, isohexadecane and, for example, the oils sold under the trade names Isopar® or Permethyl®.

Volatile oils that may also be used include volatile silicones, for instance volatile linear or cyclic silicone oils, for instance those with a viscosity≦8 centistokes (8×10⁻⁶ m²/s), and for example containing from 2 to 10 silicon atoms and for example from 2 to 7 silicon atoms, these silicones may comprise alkyl or alkoxy groups containing from 1 to 10 carbon atoms. As a volatile silicone oil that may be used in the invention, mention may be made of dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro oils such as nonafluoromethoxybutane or perfluoromethyl-cyclopentane, and mixtures thereof, may also be used.

It is also possible to use a mixture of the oils mentioned above.

A cosmetic composition according to the invention may comprise at least one volatile oil in a content ranging from about 2% to about 70% by weight, for example ranging from about 5% to about 60% by weight and for example ranging from about 10% to about 50% by weight of oil relative to the total weight of the composition.

When introduced in sufficient amount, and in combination with an ester of dimerdilinoleic acid and of polyol(s), or an ester thereof, and a film-forming agent as defined above, the volatile oil may have the advantage of being able to give an improvement in the gloss staying power and/or in the colour staying power to the cosmetic compositions according to the invention.

Non-Volatile Oils

For the purposes of the present invention, the term “non-volatile oil” means an oil with a vapour pressure of less than 0.13 Pa and for example oils of high molar mass.

A cosmetic composition according to the present invention may also comprise at least one non-volatile oil.

The non-volatile oils may be selected from the group constituted of non-volatile hydrocarbon-based oils, which may be fluorinated, and/or non-volatile silicone oils.

Non-volatile hydrocarbon-based oils also suitable for the embodiment of the invention that may be mentioned include:
hydrocarbon-based oils of animal origin,
hydrocarbon-based oils of plant origin such as phytostearyl esters, such as phytostearyl oleate, phytostearyl isostearate and lauroyl/octyldodecyl/phytostearyl glutamate, for example sold under the name Eldew PS203 by Ajinomoto, triglycerides consisting of fatty acid esters of glycerol, the fatty acids of which may have chain lengths ranging from C₄ to C₂₄, these chains possibly being linear or branched, and saturated or unsaturated; these oils are for example heptanoic or octanoic triglycerides, wheatgerm oil, sunflower oil, grapeseed oil, sesame oil, corn oil, apricot oil, castor oil, shea oil, avocado oil, olive oil, soybean oil, sweet almond oil, palm oil, rapeseed oil, cotton seed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil or musk rose oil; shea butter; or alternatively caprylic/capric acid triglycerides, for instance those sold by the company Stearineries Dubois or those sold under the names Miglyol 810®, 812® and 818′ by the company Dynamit Nobel,
hydrocarbon-based oils of mineral or synthetic origin, for instance:
   synthetic ethers containing from 10 to 40 carbon atoms;
   linear or branched hydrocarbons of mineral or synthetic origin such as petroleum jelly, polydecenes, hydrogenated polyisobutene such as Parleam, and squalane, and mixtures thereof, and for example hydrogenated polyisobutene,
   synthetic esters, for instance oils of formula R₁COOR₂ in which R₁ represents a linear or branched fatty acid residue containing from 1 to 40 carbon atoms and R₂ represents a hydrocarbon-based chain that is for instance branched, containing from 1 to 40 carbon atoms provided that R₁+R₂≧10.

The esters may be selected from the group constituted of fatty acid esters, for example:
cetostearyl octanoate, isopropyl alcohol esters, such as isopropyl myristate or isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and for example isostearyl heptanoate, alcohol or polyalcohol octanoates, decanoates or ricinoleates, for instance propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof, C₁₂ to C₁₅ alcohol benzoates, hexyl laurate, neopentanoic acid esters, for instance isodecyl neopentanoate, isotridecyl neopentanoate, isostearyl neopentanoate and octyldodecyl neopentanoate, isononanoic acid esters, for instance isononyl isononanoate, isotridecyl isononanoate and octyl isononanoate, and hydroxylated esters, for instance isostearyl lactate and diisostearyl malate;
polyol esters and pentaerythritol esters, for instance dipentaerythrityl tetrahydroxystearate/tetraisostearate;
esters of diol dimers and of diacid dimers, such as Lusplan DD-DA5® and Lusplan DD-DA7®, sold by the company Nippon Fine Chemical and described in patent application FR 03/02809;
fatty alcohols that are liquid at room temperature, with a branched and/or unsaturated carbon-based chain containing from 12 to 26 carbon atoms, for instance 2-octyldodecanol, isostearyl alcohol, oleyl alcohol, 2-hexyldecanol, 2-butyloctanol and 2-undecylpentadecanol;
higher fatty acids such as oleic acid, linoleic acid or linolenic acid, and mixtures thereof; and
dialkyl carbonates, the two alkyl chains possibly being identical or different, such as dicaprylyl carbonate sold under the name Cetiol CC® by Cognis;
non-volatile silicone oils, for instance non-volatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenylmethyldiphenyltrisiloxanes and 2-phenylethyl trimethylsiloxysilicates, and dimethicones or phenyltrimethicones with a viscosity of less than or equal to 100 Cst, and mixtures thereof;
and mixtures thereof.

The non-volatile oils may be present in a composition according to the invention in a content ranging from 5% to 90% by weight, for example from 25% to 80% by weight and for example from 40% to 70% by weight relative to the total weight of the composition.

Solid Fatty Substances

The composition according to the invention may also comprise at least one solid fatty phase selected from the group constituted of waxes and pasty fatty substances, and mixtures thereof.

The wax is solid at room temperature (25° C.), with a reversible solid/liquid change of state, having a melting point of greater than 30° C., which may be up to 200° C., a hardness of greater than 0.5 MPa and having in the solid state an anisotropic crystal organization.

It may be a hydrocarbon-based wax, a fluoro wax and/or a silicone wax and may be of animal, plant, mineral or synthetic origin.

It may be chosen, for example, from beeswax, carnauba wax, candelilla wax, paraffin waxes, hydrogenated castor oil, synthetic waxes, for instance polyethylene waxes (preferably with a molecular weight of between 400 and 600) or Fischer-Tropsch waxes, silicone waxes, for instance alkyl or alkoxy dimethicones containing from 16 to 45 carbon atoms, ceresins or ozokerites, for instance isoparaffins with a melting point of less than 40° C., such as EMW-0003 sold by the company Nippon Seirou, α-olefin oligomers, such as the polymers Performa V® 825, 103 and 260 sold by the company New Phase Technologies; ethylene-propylene copolymers, such as Performalene® EP 700, and microcrystalline waxes with a melting point of greater than 85° C., such as Hi-Mic® 1070, 1080, 1090 and 3080, sold by Nippon Seirou, and mixtures thereof.

According to one embodiment, the wax(es) used in the cosmetic compositions in accordance with the present invention may be present in a content ranging from about 5% to about 30%, for example from about 5% to about 25%, for example from about 10% to about 20% and for example from about 10% to about 15% by weight relative to the total weight of the composition.

A cosmetic composition in accordance with the present invention may also comprise at least one pasty compound.

For the purposes of the present invention, the term “pasty compound” means a fatty compound with a reversible solid/liquid change of state, and comprising at a temperature of 23° C. a liquid fraction and a solid fraction. The term “pasty substance” also means polyvinyl laurate.

For the purposes of the invention, a pasty compound may advantageously have a hardness at 20° C. ranging from 0.001 to 0.5 MPa and preferably from 0.002 to 0.4 MPa.

Among the pasty compounds that may be used in the composition according to the invention, mention may be made of lanolins and lanolin derivatives, for instance acetylated lanolins, oxypropylenated lanolins or isopropyl lanolate, and mixtures thereof. Esters of fatty acids or of fatty alcohols may also be used, for example those containing from 20 to 65 carbon atoms, for instance triisostearyl or cetyl citrate; arachidyl propionate; polyvinyl laurate; cholesterol esters, for instance triglycerides of plant origin such as hydrogenated plant oils, viscous polyesters and mixtures thereof. Triglycerides of plant origin that may be used include hydrogenated castor oil derivatives, such as Thixinr® from Rheox.

Mention may also be made of polyesters resulting from the esterification of a carboxylic acid and of an aliphatic hydroxycarboxylic acid ester. For example, Risocast® DA-L (ester derived from the esterification reaction of hydrogenated castor oil with dilinoleic acid in proportions of 2 to 1) and Risocast® DA-H (ester resulting from the esterification of hydrogenated castor oil with isostearic acid in proportions of 4 to 3) sold by the Japanese company Kokyu Alcohol Kogyo.

As pasty compounds that are advantageously suitable for formulating the cosmetic compositions in accordance with the present invention, mention may be made of hydrogenated cocoglycerides.

Mention may also be made of pasty silicone compounds such as high molecular weight polydimethylsiloxanes (PDMS) and for example those containing pendent chains of the alkyl or alkoxy type containing from 8 to 24 carbon atoms, and having a melting point of 20-55° C., for instance stearyl dimethicones, for example those sold by the company Dow Corning under the trade names DC2503® and DC25514®, and mixtures thereof.

Gloss and Gloss Staying Power

The term “gloss” is intended to denote the gloss of a composition as may be conventionally measured using a glossmeter, via methods known to those skilled in the art.

For example, the gloss may be measured using a test performed in vitro using a Muti-Gloss 268 glossmeter (Minolta, Tokyo, Japan).

Such a test may be performed on a sample of Bio-Skin synthetic skin (of the type Fat-Skin Beaulax, Tokyo, Japan) 3 cm×4.5 cm in size prepared by applying a first coat of cosmetic composition, for example a lipstick, in the direction of the length of the support, in the form of continuous strips, so as to cover an area of 2.5 cm×4 cm. A second coat of the composition is applied over the first coat in a similar manner to the first, offset widthwise by about half the width of a strip in order to obtain a uniformly covered surface.

After applying the two coats of compositions, the gloss is measured with a glossmeter by measuring the reflectance at an angle of about 60° taken relative to the perpendicular to the surface. The gloss is advantageously measured 10 minutes after applying the cosmetic composition.

The measurement is repeated, for example at least five times, and a mean is determined of the values remaining after removing the lowest value and the highest value.

The term “gloss staying power” is intended to denote the staying power of the gloss of a composition as may be measured using a glossmeter via methods known to those skilled in the art.

For example, the protocol as defined above may be followed by applying a fabric to the sample of synthetic skin comprising the two coats of cosmetic compositions. Such a fabric may be, for example, a Kleenex tissue.

The fabric may be applied using a press, for example of mass 1.5 kg, for a defined time, for example 2 seconds, in a controlled manner with a rheometer (Exponet 32 texture).

Next, the gloss of the sample is measured as defined above, and the value obtained is indicative of the staying power.

A composition according to the invention may have a gloss staying power of greater than or equal to about 4, for example greater than or equal to about 5, for example greater than about 6.

Colour Staying Power of the Composition

The term “colour staying power” is intended to denote the property of a cosmetic composition according to the invention of transferring to a minimum extent onto objects with which it may come into contact, and the property of withstanding interaction with liquids, for instance tears or sweat, or contact with food during a meal, for instance in the case, for example, of lipstick, and the property of not migrating from the initial line of the makeup, for example in the case of lipsticks, into the wrinkles and fine lines around the lips.

A film of cosmetic composition applied to the skin, mucous membranes and/or the lips may generally be impaired during contact with liquids, for instance water or drinks consumed, for example, during a meal, or alternatively oils, for instance food oils, or else sebum or saliva.

Thus, the colour staying power of a cosmetic composition may be characterized by at least one of the following parameters: the transfer resistance, the colour resistance to water and the colour resistance to oil.

Measurement of the colour resistance to oil may be an adequate parameter for characterizing a composition according to the invention, and may be performed as follows.

The measurements are taken, for example, on the inner face of the forearm, washed and dried naturally at room temperature for 5 minutes. The cosmetic composition to be tested, for example a lipstick, is applied to five areas of the inner face of the forearm. The area of skin on which the measurements are taken should be at least greater than 1 cm². In general, the measurements are taken on circular areas equal to about 3 cm in diameter.

It is necessary for about the same amount of cosmetic composition to be applied to each of the three areas. This may be checked by measuring the weight of the first and the second cosmetic composition, after each of the applications, or by preparing beforehand equivalent amounts of sample to be tested. In general, for a surface area of 1 cm², an amount equal to about 2 mg is necessary (if the surface area has a diameter of 3 cm, then an amount of about 14 mg is required).

After applying the cosmetic composition, the colour, L₁*a₁*b₁*(C₁), is measured at each of the three areas, and the mean value obtained corresponds to the initial colour of the product. The colour measurement may be performed using a Minolta colorimeter of the CR300, CM500, CM1000 or CM2000 series. The Minolta colorimeter of the CR300 series may be used.

The test of colour resistance to oil is performed by applying to the test areas about 14 mg/cm² of oil of food type onto each area of the forearm (rapeseed oil, soybean oil or sunflower oil) followed by manual massaging for a few seconds, for instance for 2 to 5 seconds or for example for 2 seconds. A thickness of a commercial white paper handkerchief such as a Kleenex tissue is then applied to the area for about 5 seconds at a force of about 100 g/f, this force possibly being applied using a DPZ-5N digital pressure dynamometer from the manufacturer Imada Co. Ltd.

The value of the colour resistance to oil, H, is equal to the difference between the mean colour L₂*a₂*b₂*(CP) of the composition remaining on the forearm after massaging with oil and application of the handkerchief, and the mean colour L₁*a₁*b₁*(C₁) initially measured,
ΔE(H)=\sqrt{(L₂*−L₁*)²+(a₂*−a₁*)²+(b₂*−b₁*)}²

The test of colour resistance to oil is a test that makes it possible to evaluate the staying power of a cosmetic composition such as a lipstick during a meal.

According to an embodiment, the colour resistance to oil of a deposit of a composition according to the invention, once spread onto a support, may be less than or equal to about 20, for example less than or equal to about 15, for example less than or equal to about 10, or less than or equal to about 8.

Dyestuff

A cosmetic composition according to the invention may also comprise at least one dyestuff.

Such a dyestuff may be chosen, for example, from water-soluble or water-insoluble, liposoluble or non-liposoluble, organic or mineral dyestuffs, for example of the type such as pigments or nacres, conventionally used in cosmetic compositions.

The term “pigments” should be understood as meaning white or coloured, mineral or organic particles that are insoluble in an aqueous solution, which are intended to colour and/or opacify the resulting film.

The pigments may be present in a proportion of from 0.5% to 30% by weight, for example from 5% to 25% by weight and for example from 10% to 20% by weight relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

It may also be a pigment having a structure that may be, for example, of sericite/brown iron oxide/titanium dioxide/silica type. Such a pigment is sold, for example, under the reference Coverleaf® NS or JS by the company Chemicals and Catalysts, and has a contrast ratio in the region of 30.

The dyestuff may also comprise a pigment having a structure that may be, for example, of silica microsphere type containing iron oxide. An example of a pigment having this structure is the product sold by the company Miyoshi under the reference PC BALL® PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPP) described in documents EP-A-542669, EP-A-787730, EP-A-787731 and WO-A-96/08537.

The term “nacres” should be understood as meaning coloured particles of any form, which may or may not be iridescent, for example produced by certain mollusks in their shell, or alternatively synthesized, and which have a colour effect via optical interference.

The nacres may be selected from the group constituted of nacreous pigments such as titanium mica coated with an iron oxide, mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica®, Flamenco® and Duochrome® (on mica base) sold by the company Engelhard, the Timiron® nacres sold by the company Merck, the Prestige® nacres on mica base sold by the company Eckart and the Sunshine® nacres on synthetic mica base sold by the company Sun Chemical.

The nacres may more particularly have a yellow, pink, red, bronze, orange, brown and/or coppery colour or glint.

As illustrations of nacres that may be used in the context of the present invention, mention may be made of gold-coloured nacres sold for example by the company Engelhard under the name Brilliant gold 212G (Timica), Gold 222C (Cloisonne), Sparkle gold (Timica), Gold 4504 (Chromalite) and Monarch gold 233×(Cloisonne); the bronze nacres sold for example by the company Merck under the names Bronze fine (17384) (Colorona) and Bronze (17353) (Colorona) and by the company Engelhard under the name Super bronze (Cloisonne); the orange nacres sold for example by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold for example by the company Engelhard under the names Nu-antique copper 340XB (Cloisonne) and Brown CL4509 (Chromalite); the nacres with a copper glint sold for example by the company Engelhard under the name Copper 340A (Timica); the nacres with a red glint sold for example by the company Merck under the name Sienna fine (17386) (Colorona); the nacres with a yellow glint sold for example by the company Engelhard under the name Yellow (4502) (Chromalite); the red-tinted nacres with a golden glint sold for example by the company Engelhard under the name Sunstone G012 (Gemtone); the pink nacres sold for example by the company Engelhard under the name Tan opale G005 (Gemtone); the black nacres with a golden glint sold for example by the company Engelhard under the name Nu antique bronze 240 AB (Timica); the blue nacres sold for example by the company Merck under the name Matte blue (17433) (Microna); the white nacres with a silvery glint sold for example by the company Merck under the name Xirona Silver; and the golden-green pinkish-orange nacres sold for example by the company Merck under the name Indian summer (Xirona), and mixtures thereof.

The cosmetic composition according to the invention may also comprise at least one water-soluble or liposoluble dye in a content ranging from 0.5% to 30% by weight and for example ranging from 5% to 25% by weight relative to the total weight of the cosmetic composition.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow. The water-soluble dyes are, for example, beetroot juice or methylene blue.

The cosmetic composition according to the invention may also contain at least one material with a specific optical effect.

This effect is different from a simple conventional hue effect, i.e. a unified and stabilized effect as produced by standard dyestuffs, for instance monochromatic pigments.

For the purposes of the invention, the term “stabilized” means lacking the effect of variability of the colour with the angle of observation or in response to a temperature change.

For example, this material may be selected from the group constituted of particles with a metallic glint, goniochromatic colouring agents, diffracting pigments, thermochromic agents, optical brighteners, and also fibres, for example interference fibres. Needless to say, these various materials may be combined so as to afford the simultaneous manifestation of two effects, or even of a novel effect in accordance with the invention.

The particles with a metallic glint that may be used in the invention may be selected from the group constituted of:
particles of at least one metal and/or of at least one metal derivative,
particles comprising a monomaterial or multimaterial organic or mineral substrate, at least partially coated with at least one layer with a metallic glint comprising at least one metal and/or at least one metal derivative, and
mixtures of the said particles.

Among the metals that may be present in the said particles, mention may be made, for example, of Ag, Au, Cu, Al, Ni, Sn, Mg, Cr, Mo, Ti, Zr, Pt, Va, Rb, W, Zn, Ge, Te and Se and mixtures or alloys thereof. Ag, Au, Cu, Al, Zn, Ni, Mo and Cr, and mixtures or alloys thereof (for example bronzes and brasses) may be useful metals.

The term “metal derivatives” is intended to denote compounds derived from metals, for example oxides, fluorides, chlorides and sulfides.

Illustrations of these particles that may be mentioned include aluminium particles, such as those sold under the names Starbrite 1200 EAC® by the company Siberline and Metalure® by the company Eckart.

Mention may also be made of metal powders of copper or of alloy mixtures such as the references 2844 sold by the company Radium Bronze, metallic pigments, for instance aluminium or bronze, such as those sold under the names Rotosafe® 700 from the company Eckart, silica-coated aluminium particles sold under the name Visionaire Bright Silver® from the company Eckart, and metal alloy particles, for instance the silica-coated bronze (alloy of copper and zinc) powders sold under the name Visionaire Bright Natural Gold® from the company Eckart.

They may also be particles comprising a glass substrate, for instance those sold by the company Nippon Sheet Glass under the name Microglass Metashine®.

The goniochromatic colouring agent may be chosen, for example, from interference multilayer structures and liquid-crystal colouring agents.

Examples of symmetrical interference multilayer structures that may be used in compositions produced in accordance with the invention are, for example, the following structures: Al/SiO₂/Al/SiO₂/Al, pigments having this structure being sold by the company Dupont de Nemours; Cr/MgF₂/Al/MgF₂/Cr, pigments having this structure being sold under the name Chromaflair® by the company Flex; MoS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl® by the company BASF; MoS₂/SiO₂/mica oxide/SiO₂/MoS₂; Fe₂O₃/SiO₂/mica oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona® by the company Merck (Darmstadt). By way of example, these pigments may be the pigments of silica/titanium oxide/tin oxide structure sold under the name Xirona Magic® by the company Merck, the pigments of silica/brown iron oxide structure sold under the name Xirona Indian Summer® by the company Merck and the pigments of silica/titanium oxide/mica/tin oxide structure sold under the name Xirona Caribbean Blue® by the company Merck. Mention may also be made of Infinite Colors® pigments from the company Shiseido. Depending on the thickness and the nature of the various coats, different effects are obtained. Thus, with the structure Fe₂O₃/SiO₂/Al/SiO₂/Fe₂O₃, the colour changes from green-golden to red-grey for SiO₂ layers of from 320 to 350 nm; from red to golden for SiO₂ layers of from 380 to 400 nm; from violet to green for SiO₂ layers of from 410 to 420 nm; from copper to red for SiO₂ layers of from 430 to 440 nm.

Examples of pigments with a polymeric multilayer structure that may be mentioned include those sold by the company 3M under the name Color Glitter®.

Examples of liquid-crystal goniochromatic particles that may be used include those sold by the company Chenix and also the product sold under the name Helicone® HC by the company Wacker.

According to one embodiment, a composition according to the invention may comprise at least one dyestuff chosen, for example, from organic dyestuffs and inorganic dyestuffs, such as pigments and nacres, and materials with a specific optical effect, and mixtures thereof.

According to one embodiment, a composition according to the invention may comprise not more than 30% by weight of dyestuff relative to the total weight of the composition.

Fillers

The cosmetic compositions in accordance with the invention may also comprise at least one filler, of organic or mineral nature, which for example makes it possible to give them improved stability with regard to exudation.

The term “filler” should be understood as meaning colourless or white solid particles of any form, which are in an insoluble form and dispersed in the medium of the composition. Being of mineral or organic nature they make it possible to impart body or rigidity to the composition, and/or softness, a matt effect and uniformity to the makeup.

The fillers used in the compositions according to the present invention may be of lamellar, globular or spherical form, fibres or in any other form intermediate between these defined forms.

The fillers according to the invention may or may not be surface-coated, and they may be surface-treated with silicones, amino acids, fluoro derivatives or any other substance that promotes the dispersion and compatibility of the filler in the composition.

For the purposes of the present invention, the terms “mineral fillers” and “inorganic fillers” are used interchangeably.

Among the mineral fillers that may be used in the compositions according to the invention, mention may be made of talc, mica, silica, trimethyl siloxysilicate, kaolin, bentone, precipitated calcium carbonate, magnesium carbonate, magnesium hydrogen-carbonate, hydroxyapatite, boron nitride, hollow silica microspheres (Silica Beads® from Maprecos), glass or ceramic microcapsules, silica-based fillers, for instance Aerosil® 200 or Aerosil® 300; Sunsphere® L-31 and Sunsphere® H-31 sold by Asahi Glass; Chemicelen® sold by Asahi Chemical; composites of silica and of titanium dioxide, for instance the TSG series sold by Nippon Sheet Glass, and mixtures thereof.

Among the organic fillers that may be used in the compositions according to the invention, mention may be made of polyamide powders (Nylon® Orgasol from Atochem), poly-β-alanine powder and polyethylene powder, polytetrafluoroethylene powders (Teflon®), N-lauroyl-L-lysine, starch, tetrafluoroethylene polymer powders, hollow polymer microspheres such as Expancel® (Nobel Industrie), precipitated calcium carbonate, magnesium carbonate, magnesium hydrogen carbonate, metal soaps derived from organic carboxylic acids containing from 8 to 22 carbon atoms and for example from 12 to 18 carbon atoms, for example zinc stearate, magnesium stearate or lithium stearate, zinc laurate or magnesium myristate, Polypore® L 200 (Chemdal Corporation), silicone resin microbeads (for example Tospearl® from Toshiba), polyurethane powders, for example powders of crosslinked polyurethane comprising a copolymer, the said copolymer comprising trimethylol hexyllactone. For example, it may be a polymer of hexamethylene diisocyanate/trimethylol hexyllactone. Such particles are for instance commercially available, for example, under the name Plastic Powder D-400® or Plastic Powder D-800® from the company Toshiki, and mixtures thereof.

A filler may be present in a cosmetic composition in accordance with the invention in a proportion of from 0.5% to 40% by weight and for example from 5% to 30% by weight of filler relative to the total weight of the composition.

A filler that is suitable for the invention may be, for example, a filler whose mean particle size is less than 100 μm and for example between 1 and 50 μm, for example between 4 and 20 μm.

Additives

The cosmetic compositions according to the invention may, furthermore, also comprise any additive usually used in the field under consideration, selected from the group constituted of surfactants, gelling agents, semi-crystalline polymers, gums, antioxidants, essential oils, preserving agents, fragrances, neutralizers, moisturizers, antiseptics, vitamins such as vitamin B3 or E and derivatives thereof, and anti-UV agents, and mixtures thereof.

According to one embodiment, a subject of the present invention is the use of at least one ester of dimerdilinoleic acid and of polyol(s) or an ester thereof, the viscosity of which, measured at about 25° C., is greater than or equal to about 20 000 mPa·s, in combination with at least one volatile oil and at least one film-forming agent, for the preparation of a cosmetic composition for caring for and/or making up the skin and/or the lips, which has improved gloss staying power, or even improved colour staying power.

According to an embodiment, the ester, the film-forming agent and the volatile oil may be, for example, as defined above.

A composition in accordance with the invention may be prepared in the usual manner by a person skilled in the art. It may thus be in cast form, for example in the form of a stick or wand, or in the form of a soft paste in a heating bag or in the form of dishes that may be used by direct contact or with a sponge.

For example, it may constitute a cast foundation, a cast makeup rouge or eyeshadow, which is for example coloured, a lipstick, a lip gloss or a concealer product.

A composition according to the invention may be obtained by heating the various constituents to the melting point of the highest-melting waxes, followed by pouring the molten mixture into a mould (crucible or finger stall). It may also be obtained by extrusion, as described in patent application EP-A-0 667 146.

The present invention also relates to a process for making up and/or caring for the skin and/or the lips, comprising at least one step that consists in applying a composition in accordance with the invention to at least part of a support, as for example a synthetic support.

According to another of its aspects, a subject of the present invention is a synthetic support comprising a composition in accordance with the invention on at least part of its surface.

A cosmetic composition according to the invention may be in the form of a lip makeup and/or care composition, for example a lipstick, a lip balm or a gloss.

For the purposes of the present invention, and unless otherwise indicated, the word “one” should be understood as meaning “at least one”.

The examples of compositions below are given as illustrations and with no limiting nature on the invention.

### EXAMPLE

Lipstick

| | % mass |
|---|---|
| Hexadecene/PVP copolymer | 1.82 |
| Polyglyceryl-2 triisostearate | 1.00 |
| Polyglyceryl-2 isostearate/dimerdilinoleate | 4.00 |
| copolymer | |
| (HAILUCENT ISDA (HAI)) | |
| Dimerdilinoleyl dimerdilinoleate | 2.00 |
| (LUSPLAN DD-DA7 (NFC)) | |
| Bis-behenyl/isostearyl/phytosteryl | 1.00 |
| dimerdilinoleyl dimerdilinoleate | |
| (Plandool-G (NFC)) | |
| Hydrogenated polyisobutene | 11.00 |
| Isononyl isononanoate | 2.70 |
| Neopentyl glycol dicaprate | 2.27 |
| Phenyl trimethicone | 2.70 |
| Isododecane | 1.82 |
| D5 | 36.32 |
| PSPA | 3.64 |
| MQ resin | 5.46 |
| BHT | 0.05 |
| Polyethylene (M: 500) | 13.64 |
| Candelilla wax | 1.82 |
| Titanium dioxide | 0.18 |
| D & C RED 7 W 012 C | 0.41 |
| Iron oxides | 0.86 |
| YELLOW 5 LAKE | 0.77 |
| BLUE 1 LAKE | 0.18 |
| Ethylene glycol dimethacrylate/lauryl | 0.91 |
| methacrylate copolymer | |
| N-lauroyl L-lysine | 0.91 |
| Silica | 0.45 |
| Mica-coated iron oxide | 2.55 |
| Calcium and aluminium borosilicate- | 0.45 |
| coated titanium dioxide | |
| Mica-coated titanium dioxide | 0.91 |
| Simethicone | 0.18 |
| Total | 100 |

Procedure

An oily phase is prepared by mixing, while heating (about 95° C.), part of the oils, the film-forming agent and the polyglyceryl-2 isostearate dimerdilinoleate copolymer (Hailucent ISDA).

The oily phase thus prepared is stirred at about 95° C. and the fillers are added to the mixture.

The waxes, the pigments in the form of a pigmentary paste, prepared by mixing the dyestuffs with the polyglyceryl-2 triisostearate, the remaining oils, the simethicone and the bis-behenyl/isostearyl/phytosteryl dimerdilinoleyl dimerdilinoleate (Plandool-G) are then added to the mixture.

The mixture thus obtained is then poured into a lipstick mould and left to cool until a solid composition is obtained.

The gloss staying power and the colour staying power of the composition are measured as described previously.

The gloss staying power is 7.1 and the colour staying power is 5.4.

1. An anhydrous cosmetic composition for caring for and/or making up the skin and/or the lips, comprising at least one ester of dimerdilinoleic acid and of polyol(s) or an ester thereof, the viscosity of which, measured at about 25° C., is greater than or equal to about 20 000 mPa·s, at least one film-forming agent and at least one volatile oil.

2. The composition according to claim 1, in which the said ester has a molecular weight ranging from about 2000 to about 25 000 g/mol.

3. The composition according to claim 1, in which the polyol is a diol.

4. The composition according to claim 3, in which the ester comprises an alternating sequence of dimerdilinoleate residue(s) and of residue(s) related to the said diol(s).

5. The composition according to claim 3, in which the diol is selected from the group constituted of a fatty alcohol dimer, a monoglycerol or polyglycerol, a C₂-C₄ monoalkylene or polyalkylene glycol, 1,4-butanediol and pentaerythritol.

6. The composition according to claim 4, in which the diol is selected from the group constituted of a fatty alcohol dimer, a monoglycerol or polyglycerol, a C₂-C₄ monoalkylene or polyalkylene glycol, 1,4-butanediol and pentaerythritol.

7. The composition according to claim 5, in which the fatty alcohol dimer is the product of hydrogenation of a fatty acid dimer obtained by dimerization of a C₈-C₃₄ unsaturated fatty acid.

8. The composition according to claim 6, in which the fatty alcohol dimer is the product of hydrogenation of a fatty acid dimer obtained by dimerization of a C₈-C₃₄ unsaturated fatty acid.

9. The Composition according to claim 4, in which each of the two ends of the said sequence respectively bears a unit OR′ and OR″, with R′ and R″ representing, independently of each other, a hydrogen atom or OR′ and OR″ representing, independently of each other, a C₂-C₃₆ hydrocarbon-based monoalcohol residue.

10. The composition according to claim 9, in which R′ and R″ both represent a hydrogen atom.

11. The composition according to claim 9, in which OR′ and OR″ both represent an identical or different hydrocarbon-based monoalcohol residue.

12. The composition according to claim 1, comprising an ester of general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15, at least one film-forming agent and at least one volatile oil.

13. The composition according to claim 12, in which OR₂O represents a dimerdilinoleyl residue.

14. The composition according to claim 12, in which OR₃ represents a hydrocarbon-based monoalcohol residue selected from the group constituted of behenyl, isostearyl and phytosteryl residues, and mixtures thereof.

15. The composition according to claim 1, comprising an ester of general formula (II) below: 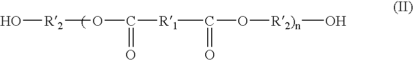
in which:
n is an integer ranging from 1 to 15,
COR′₁CO represents a dimerdilinoleate residue,
OR′₂O represents a diglyceryl residue of general formula (III) below: 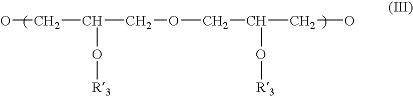
in which:
R′₃ represents H or OR′₃ represents a fatty acid residue, at least one film-forming agent and at least one volatile oil.

16. The composition according to claim 15, in which the fatty acid residue featured by OR′₃ is an isostearyl residue.

17. The composition according to claim 1, in which the said ester is selected from the group constituted of the esters having the following INCI nomenclature: polyglyceryl-2 isostearate/dimerdilinoleate copolymer, bisbehenyl/isostearyl/phytosteryl dimerdilinoleyl dimerdilinoleate, and mixtures thereof.

18. The composition according to claim 1, wherein it comprises from 0.1% to 80% by weight of the said ester relative to the total weight of the composition.

19. The composition according to claim 1, in which the film-forming agent is a film-forming polymer.

20. The composition according to claim 9, in which the film-forming polymer is selected from the group constituted of film-forming polymers that are soluble in an organic liquid medium, film-forming polymers that are dispersible in an organic liquid medium, film-forming polymers in the form of aqueous dispersions of polymer particles, water-soluble film-forming polymers, and mixtures thereof.

21. The composition according to claim 1, in which the film-forming agent is present in a content ranging from about 0.1% to about 60% by weight relative to the total weight of the composition.

22. The composition according to claim 1, also comprising at least one auxiliary film-forming agent.

23. The composition according to claim 1, in which the said volatile oil is selected from the group constituted of:
hydrocarbon-based oils containing from 8 to 16 carbon atoms,
linear or cyclic silicone oils,
volatile fluoro oils, and
mixtures thereof.

24. The composition according to claim 1, in which the volatile oil is present in a content ranging from about 2% to about 70% by weight of oil relative to the total weight of the composition.

25. The composition according to claim 1, also comprising at least one non-volatile oil selected from the group constituted of hydrocarbon-based oils of animal origin, hydrocarbon-based oils of plant origin and hydrocarbon-based oils of mineral or synthetic origin, and mixtures thereof.

26. The composition according to claim 1, wherein it also comprises at least one solid fatty phase selected from the group constituted of pasty fatty substances and waxes, and mixtures thereof.

27. The composition according to claim 1, also comprising at least one dyestuff.

28. The composition according to claim 27, in which the dyestuff is selected from the group constituted of organic dyestuffs and inorganic dyestuffs.

29. The composition according to claim 1, wherein it is in the form of a suspension, a dispersion, a solution, a gel, an emulsion, or a multiple emulsion (W/O/W or polyol/O/W or O/W/O) or in the form of a cream, a stick, a paste, a mousse, a vesicular dispersion, a two-phase or multi-phase lotion or a powder.

30. The composition according to claim 1, wherein it is a lip makeup and/or care composition.

31. A cosmetic composition for caring for and/or making up the skin and/or the lips, comprising at least one ester of general formula (I) below:
R₃—OCO—R₁(—COO—R₂—OCO—R₁)ₙ—COO—R₃ (I)
in which:
COR₁CO represents a dimerdilinoleate residue,
OR₂O represents a fatty alcohol dimer residue,
OR₃ represents a hydrocarbon-based monoalcohol residue, and
n is an integer ranging from 1 to 15,
at least one film-forming agent and at least one volatile oil.

32. The composition according to claim 31, in which OR₂O represents a dimerdilinoleyl residue.

33. The composition according to claim 31, in which OR₃ represents a hydrocarbon-based monoalcohol residue selected from the group constituted of behenyl, isostearyl and phytosteryl residues, and mixtures thereof.

34. The composition according to claim 31, wherein it is anhydrous.

35. A cosmetic composition for caring for and/or making up the skin and/or the lips, comprising at least one ester of general formula (II) below: 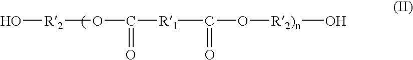
in which:
n is an integer ranging from 1 to 15,
COR′₁CO represents a dimerdilinoleate residue,
OR′₂O represents a diglyceryl residue of general formula (III) below: 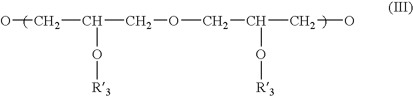
in which:
R′₃ represents H or OR′₃ represents a fatty acid residue, at least one film-forming agent and at least one volatile oil.

36. The composition according to claim 35, in which the fatty acid residue featured by OR′₃ is an isostearyl residue.

37. The composition according to claim 35, wherein it is anhydrous.

38. A method for the preparation of a cosmetic composition for caring for and/or making up the skin and/or the lips, which has improved colour staying power comprising at least a step of combining at least one ester of dimerdilinoleic acid and of polyol(s), or an ester thereof, the viscosity of which, measured at 25° C., is greater than or equal to 20 000 mPa·s, with at least one volatile oil and at least one film-forming agent.

39. The method for the preparation of a cosmetic composition for caring for and/or making up the skin and/or the lips, which has improved colour staying power comprising at least a step of combining at least one ester of dimerdilinoleic acid and of polyol(s), or an ester thereof, the viscosity of which, measured at 25° C., is greater than or equal to 20 000 mPa·s, with at least one volatile oil and at least one film-forming agent, in which the said ester is as defined according to claim 1.

40. The method for the preparation of a cosmetic composition for caring for and/or making up the skin and/or the lips, which has improved colour staying power comprising at least a step of combining at least one ester of dimerdilinoleic acid and of polyol(s), or an ester thereof, the viscosity of which, measured at 25° C., is greater than or equal to 20 000 mPa·s, with at least one volatile oil and at least one film-forming agent, in which the film-forming agent is as defined according to claim 19.

41. The method for the preparation of a cosmetic composition for caring for and/or making up the skin and/or the lips, which has improved colour staying power comprising at least a step of combining at least one ester of dimerdilinoleic acid and of polyol(s), or an ester thereof, the viscosity of which, measured at 25° C., is greater than or equal to 20 000 mPa·s, with at least one volatile oil and at least one film-forming agent, in which the volatile oil is as defined according to claim 23.

42. A process for making up and/or caring for the skin and/or the lips, comprising at least one step that consists in applying to at least a portion of the said support a composition as defined according to claim 1.
